(19) United States
(12) Patent Application Publication
(10) Pub. No.: US 2015/0228470 A1
(43) Pub. Date: Aug. 13, 2015

(54) RADIATOR UNIT FOR GENERATING ULTRAVIOLET RADIATION AND METHOD FOR ITS PRODUCTION

(71) Applicant: Heraeus Noblelight GmbH, Hanau (DE)

(72) Inventor: Hector Julian Ruiz, Hanau (DE)

(73) Assignee: Heraeus Noblelight GmbH (a German Corporation), Hanau (DE)

(21) Appl. No.: 14/428,405

(22) PCT Filed: Sep. 23, 2013
(86) PCT No.: PCT/EP2013/069718

§ 371 Date: Mar. 16, 2015

(30) Foreign Application Priority Data
Oct. 18, 2012 (DE) 10 2012 109 930.8

(51) Int. Cl. H01J 61/35; C02F 1/32; A23L 3/28

(52) U.S. Cl. CPC H01J 61/35; A23L 3/28; C02F 1/325

(57) ABSTRACT

Known radiator units for generating ultraviolet radiation, particularly for use in food processing or for the treatment of water, have a UV radiator having a radiator tube made of quartz glass or a UV radiator surrounded by a cylindrical jacket tube made of quartz glass having a radiator tube made of quartz glass. Starting from this background, in order to provide a radiator unit for generating ultraviolet radiation, which is suitable for emitting a high radiation power and is also simple and economical to produce, a contaminant- and water-repellent coating is applied to the radiator tube and/or the jacket tube. This coating is generated by use of silicon dioxide or titanium dioxide nano-particles.

The present invention relates to a radiator unit for generating ultraviolet radiation, in particular for use in food processing or for the treatment of water, comprising a UV radiator having a radiator tube made of quartz glass or a UV radiator surrounded by a cylindrical jacket tube made of quartz glass having a radiator tube made of quartz glass.

The present invention also relates to a method for the production of the radiator unit.

### PRIOR ART

Possible fields of use for radiator units are, for example, the treatment and disinfection of water, the cleaning and disinfection of gases or gas mixtures, particularly air, as well as the disinfection of surfaces.

Such radiator units comprise a UV radiator having a radiator tube made of quartz glass; they are used, for example, in water treatment plants, ventilation systems, and exhaust devices for gases or air treatment plants. Depending on the purpose of use, such a radiator unit can contribute, for example, to the killing of microorganisms, to the elimination of arising odors, or to the decomposition of contaminants.

Radiator units are often used in the processing of food. They are used both in the industrial production of food and also in institutional kitchens or in the household area.

The processing of food produces cooking and baking fumes that are also designated as waste steam. They contain, in addition to water vapor, a plurality of particulate matter and odorous substances, particularly fat.

To neutralize the odors that occur during the processing of food (deodorization) and simultaneously to reduce a deposition of particulate material, for example, in ventilation systems or air exhaust devices, in addition to mechanical filters, radiator units for generating ultraviolet radiation are also used. The use of this UV radiator unit permits a chemical decomposition of odorous substances and particulate material.

To guarantee efficient irradiation with ultraviolet radiation, the radiator units are usually arranged so that the waste steam flows around the units. Therefore, during the operation of the radiator unit, dust particles or contaminant material, in particular fat deposits, can be deposited on the radiator tube. These contaminants absorb the UV radiation emitted by the UV radiator, so that the transparency of the radiator tube and thus the efficiency of the UV irradiation decreases with increasing operating time. This has the result that the radiator tube must be regularly cleaned or the radiator must be replaced. To reduce contamination of the radiator tube, it is known to install mechanical filters in front of the radiator unit. However, even these filters only lead to partial separation of contaminants. In addition, the provision of mechanical filters requires regular replacement of the filters and therefore is intensive in terms of time and costs.

In addition, in many radiator units, particularly in those that are used for the treatment of fluids, the UV radiator is often protected from contamination, such that it is arranged in a jacket tube made of quartz glass. Due to the jacket tube, the fluid does not flow directly around the radiator tube, so that deposits of contaminant material on the radiator tube are reduced.

For example, from WO 2008/059503 A1 a system for the sterilization of fluids by ultraviolet radiation is known, which has a flow channel. Within the flow channel, several radiators surrounded by a jacket tube are arranged perpendicular to the direction of flow.

However, with the use of a jacket tube, the jacket tube itself is exposed to contamination, so that the transmission properties of the jacket tube and thus the efficiency of the radiator power are negatively affected as a function of the degree of contamination. In addition, biofilms can form on the jacket tube, which can also negatively affect the transmission of ultraviolet radiation, so that the jacket tubes also must be cleaned at regular intervals expensively by machine or by hand.

### TECHNICAL FIELD

The invention is therefore based on the problem of providing a radiator unit for generating optical radiation, which is suitable for emitting a high radiation power over a long operating period and which is also simple and economical to produce.

The invention is also based on the problem of providing a method for producing such a radiator unit.

### General Description of the Invention

With regard to the radiator unit, this problem is solved according to the invention, starting from a radiator unit for generating ultraviolet radiation of the type mentioned in the introduction, such that a contaminant and water-repellent coating, which is generated by use of silicon dioxide or titanium dioxide nanoparticles, is deposited on the radiator tube and/or the jacket tube.

A coating generated from silicon dioxide or titanium dioxide nanoparticles has, in particular, a high transparency for ultraviolet radiation. Therefore, it is suitable for coating of the jacket tube or radiator tube. Such a coating does not significantly negatively affect the radiation power of the radiator.

In addition, due to its chemical properties, such a coating can be permanently deposited on a quartz glass surface. A coating generated from silicon dioxide or titanium dioxide nanoparticles has a high degree of UV stability, good abrasion resistance, and good temperature resistance. It also provides a high degree of chemical stability.

According to the invention, the coating is deposited on the radiator tube and/or a jacket tube surrounding the radiator tube. Below, for simplifying the description, instead of the terms radiator tube and jacket tube, the general term tube will be used, with the associated description extending to both variants.

The coating covers the tube completely or partially. Preferably, coating is deposited on an outer surface of the tube.

Tubes made of quartz glass can have a slightly rough surface that basically promotes a deposition of contaminant particles. A coating having nanoparticles is therefore particularly suitable for filling out unevenness in the quartz glass surface. By depositing a coating made of nanoparticles, the surface roughness is reduced, so that a smoother surface is obtained on which contaminant particles can adhere less easily.

Furthermore, the physical and chemical properties of the silicon dioxide or titanium dioxide nanoparticles contribute to stopping the adsorption and deposition of contaminant particles. Thus, a tube surface coated according to the invention has, compared with an uncoated tube surface, a higher degree of hydrophilicity, whereby the deposition of lipophilic contaminant particles is made more difficult.

A radiator unit having a coated jacket tube or radiator tube therefore can be operated without cleaning over a long time period with a high radiation power. The lengthened cleaning intervals enable an easier and more economical operation of the radiator unit.

In one advantageous embodiment of the radiator unit according to the invention, it is provided that the coating comprises no organic substances.

The coating of the jacket tube or the radiator tube is exposed to continuous irradiation with ultraviolet radiation during the operation of the radiator unit. Irradiation of organic substances with ultraviolet radiation, however, promotes their decomposition and leads to a short service life of the coating. A coating having long service life is obtained if the coating comprises exclusively inorganic substances.

In one advantageous embodiment of the radiator unit according to the invention, it is provided that the coating has a surface having an average roughness Rₐ of less than 0.05 μm.

The average roughness Rₐ is defined as a perpendicular parameter according to DIN EN ISO 4288:1988. It indicates the average distance of a measurement point with respect to a center line of a surface profile. A surface having a roughness of greater than 0.05 μm has only limited water and contaminant-repellent properties. Therefore, it has proven effective if the average roughness Rₐ of the coated surface is less than 0.05 μm.

It has proven advantageous if the silicon dioxide nanoparticles have an average particle size in the range from 10 nm to 75 nm.

Silicon nanoparticles having an average particle size in the range from 10 nm to 75 nm are simple and economical to produce. They are particularly suitable for balancing out unevenness on a quartz glass surface.

It has proven effective if the titanium dioxide nanoparticles have an average particle size between 10 nm and 80 nm.

Titanium dioxide nanoparticles having an average particle size between 10 nm and 80 nm are simple and economical to produce. The average particle size of the nanoparticles influences the surface structure of the coating. A coating having titanium dioxide nanoparticles having an average particle size of greater than 80 nm has a relatively coarse surface structure. Nanoparticles having an average particle size of less than 10 nm are complicated and expensive to process.

In one advantageous embodiment of the radiator unit, the average layer thickness of the coating is between 60 nm and 150 nm.

The layer thickness of the coating influences the degree of transmission of the radiator tube or jacket tube. A uniform coating having an average layer thickness of less than 60 nm can be produced only with complicated and expensive methods. Coatings having an average layer thickness of greater than 150 nm can easily peel or flake and have a shorter service life.

It has proven favorable if the radiator tube and/or the jacket tube has a surface having an average roughness Rₐ in the range between 0.01 μm and 1 μm, on which the coating is deposited.

The adhesion of the coating on the radiator tube/jacket tube surface is influenced by the average roughness of the surface. A radiator tube having an average roughness Rₐ of less than 0.01 μm has a minimal surface structure and leads to poorer adhesion of the coating. A surface having an average roughness Rₐ of greater than 1 μm requires a comparatively large layer thickness of the smoothening coating.

In another preferred embodiment it is provided that the radiator tube has an emission surface that is completely provided with the coating.

In a radiator tube having a completely coated emission surface, the entire emission surface has water and contaminant-repellent properties. Such a radiator tube contributes to a uniform radiation power of the radiator over the entire period of use of the radiator.

With respect to the production process, this problem is solved according to the invention, starting from a method of the type mentioned in the introduction, in that the method comprises the following processing steps:
(a) Deposition of an alcoholic dispersion of silicon dioxide or titanium dioxide nanoparticles on the outer wall under formation of a dispersion layer, wherein the alcoholic dispersion comprises 20 vol. % to 60 vol. % ethanol, each with respect to the volume of the dispersion,
(b) Curing of the dispersion layer under formation of the coating.

For the coating, an alcoholic dispersion of silicon dioxide or titanium dioxide nanoparticles is deposited on the outer wall of the radiator tube or the jacket tube. In addition to ethanol in a concentration range from 20 vol. % to 60 vol. %, the dispersion can also contain other volatile solvents, for example methanol, isopropanol, or mixtures thereof. Ethanol has a certain degree of hydrophilicity and can also be mixed with lipophilic substances to a limited extent. In addition, the boiling point of ethanol is 78° C. Therefore, an ethanol dispersion already enables drying of the dispersion at low temperatures, for example at room temperature. After the evaporation of the solvent, the silicon dioxide nanoparticles link together to form a tight lattice. In contrast, titanium dioxide nanoparticles form a coating made of predominantly discrete titanium dioxide particles.

In one preferred modification of the method, it is provided that the alcoholic dispersion comprises 0.25 vol. % to 1.5 vol. % 2-butanone.

2-butanone has a boiling point of 80 C and is a good solvent for lipophilic substances. An addition of 2-butanone to the dispersion increases the lipophilicity of the dispersing agent.

### EMBODIMENT

The invention will be described in more detail below with reference to an embodiment and a drawing with two figures. Shown in detail in schematic representation are:

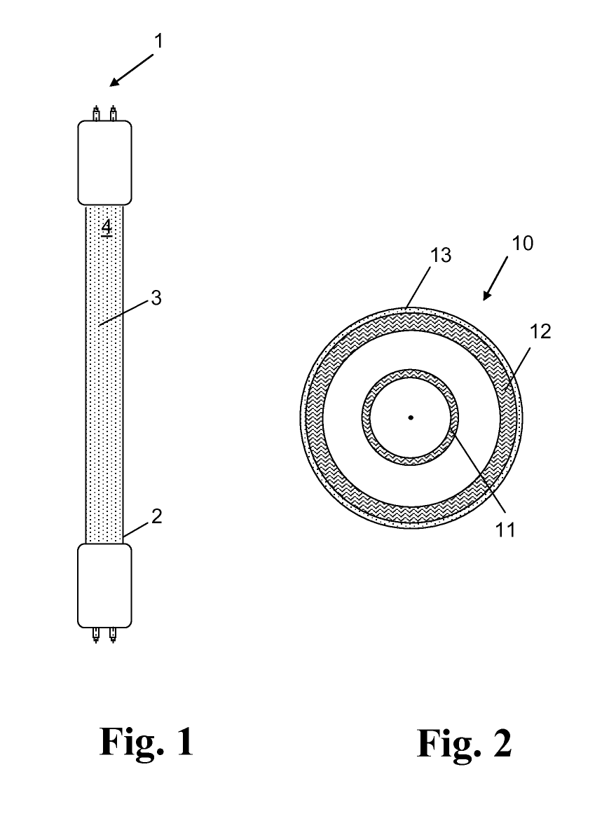

FIG. 1 an embodiment of the radiator unit according to the invention having a coated radiator tube in side view, and

FIG. 2 a second embodiment of the radiator unit according to the invention with a coated jacket tube in cross section.

FIG. 1 shows schematically an embodiment of the radiator unit according to the invention for generating ultraviolet radiation, which is assigned overall the reference numeral 1. The radiator unit 1 is suitable for use in institutional kitchens, particularly for reducing occurring odors or for preventing fat deposits, as well as odorous substances and particulate matter of waste steam from cooking and baking.

The radiator unit 1 comprises a UV radiator 2 having a radiator tube 3 made of quartz glass. The UV radiator 2 is distinguished by a nominal output of 500 W at a nominal lamp current of 2.5 A, an illuminated length of 1000 mm and a light tube outer diameter of 24 mm. A water and contaminant-repellent coating 4 is deposited on the outer 5 wall of the radiator tube 3, wherein the coating 4 completely covers the emission surface of the radiator tube 3. The coating 4 is free of organic substances.

For generating the coating on the outer wall of the radiator tube 3, an ethanol dispersion of silicon dioxide nanoparticles uses the following composition: 50 vol. % ethanol, 49 vol. % silicon dioxide nanoparticles (average particle size 50 nm), 1 vol. % 2-butanone. The ethanol dispersion is manually deposited on the outer wall of the radiator tube 3, wherein the outer wall has an average roughness Rₐ of 0.25 μm. The dispersion can alternatively also be sprayed onto the outer wall. Then, the radiator tube 3 is dried for 24 hours at room temperature under formation of the coating 4. The coated radiator tube 3 has an average roughness Rₐ of 0.02 μm. The layer thickness of the coating 4 is 120 nm.

In an alternative embodiment of the radiator unit according to the invention, the coating 4 is made of titanium dioxide nanoparticles having an average particle size of 75 nm.

FIG. 2 shows in cross section a second embodiment of the radiator unit 10 according to the invention having a cylindrical radiator 11, surrounded by a jacket tube 12 made of quartz glass. The radiator unit 10 is suitable for use in a water treatment plant (not shown).

On the cylindrical jacket tube 12, a contaminant and water-repellent coating 13 generated by use of silicon dioxide nanoparticles is deposited. The surface of the coated radiator tube has an average roughness of 0.007 μm.

### Example 1

For comparison purposes, a kitchen exhaust hood having a radiator unit according to the invention from FIG. 1 and another kitchen exhaust hood having a structurally identical, conventional radiator unit were operated. Then the transparency of the lamp tube was evaluated visually. The results of these tests are summarized in the following tables:

Results 1

| Lamp type | Operating period | Optical testing |
|---|---|---|
| Radiator tube having | 3 months | Clear, transparent radi- |
| half-side coating | (900 operating hours) | ator tube |
| Standard | 3 months | Milky, cloudy radiator |
| | (900 operating hours) | tube |

Results 2

| Lamp type | Operating period | Optical testing |
|---|---|---|
| Radiator tube having | 7 months | Clear, transparent |
| half-side coating | (2100 operating hours) | radiator tube, some |
| | | cloudy spots |
| Standard | 7 months | Milky, cloudy radiator |
| | (2100 operating hours) | tube |

With longer operating periods, radiator units having a coated radiator tube also exhibit no or only slight contaminant deposits. The radiator tubes having a coating are much clearer and transparent even after over 2000 operating hours.

### Example 2

For comparison purposes, a quartz plate was partially coated with silicon dioxide nanoparticles and the contact angle with water was measured. The results of these tests are summarized in the following table:

| | Contact angle after | Contact angle after |
|---|---|---|
| Quartz plate | 1 hour (at 120 C.) | 24 hours (at 120 C.) |
| Coated | 60° | 62° |
| Uncoated | 25° | 30° |

1-10. (canceled)

11. A radiator unit for generating ultraviolet radiation, the radiator unit comprising a UV radiator having a quartz glass tube selected from a UV radiator tube and a cylindrical jacket tube surrounding a UV radiator tube, and a contaminant- and water-repellent coating deposited on the quartz glass tube, wherein the coating comprises nanoparticles of silicon dioxide or titanium dioxide.

12. The radiator unit according to claim 11, wherein the coating comprises no organic substances.

13. The radiator unit according to claim 11, wherein the coating has a surface having an average roughness Rₐ of less than 0.05 μm.

14. The radiator unit according to claim 11, wherein the silicon dioxide nanoparticles have an average particle size in a range from 1 nm to 75 nm.

15. The radiator unit according to claim 11, wherein the titanium dioxide nanoparticles have an average particle size between 1 nm and 80 nm.

16. The radiator unit according to claim 11, wherein the coating has an average layer thickness between 60 nm and 150 nm.

17. The radiator unit according to claim 11, wherein the quartz glass tube has a surface having an average roughness Rₐ in a range between 0.01 μm and 1 μm, on which the coating is deposited.

18. The radiator unit according to claim 11, wherein the radiator tube has an emission surface completely covered with the coating.

19. The radiator unit according to claim 11, wherein the generated ultraviolet radiation is used in food processing or for treatment of water.

20. A method for producing a radiator unit according to claim 11, the method comprising providing the radiator tube or the jacket tube made of quartz glass and generating a coating on at least a portion of an outer wall of the quartz glass tube, the generating comprising the following processing steps:
(a) depositing an alcoholic dispersion of silicon dioxide or titanium dioxide nanoparticles on the outer wall under formation of a dispersion layer, wherein the alcoholic dispersion comprises 20 vol. % to 60 vol. % ethanol, based on the volume of the dispersion, and
(b) curing the dispersion layer under formation of the coating.

21. The method according to claim 20, wherein the alcoholic dispersion comprises 0.25 vol. % to 1.5 vol. % 2-butanone.
